(19) United States
(12) Patent Application Publication
(10) Pub. No.: US 2026/0200818 A1
(43) Pub. Date: Jul. 16, 2026

(54) PROCESS FOR PREPARING ISOPROPANOL IN HIGH YIELDS AND WITH A HIGH DEGREE OF PURITY

(71) Applicant: VERSALIS S.P.A., San Donato Milanese (MI) (IT)

(72) Inventors: Giovanni Antonio FOIS, Mantova (MN) (IT); Riccardo FELISARI, Mantova (MN) (IT)

(21) Appl. No.: 19/136,404

(22) PCT Filed: Dec. 4, 2023
(86) PCT No.: PCT/IB2023/062188

§ 371 Date: Jun. 6, 2025

(30) Foreign Application Priority Data
Dec. 6, 2022 (IT) 102022000025110

(51) Int. Cl. C07C 29/145; B01J 23/72

(52) U.S. Cl. CPC C07C 29/145; B01J 23/72

(57) ABSTRACT

A process for producing isopropanol through a catalytic hydrogenation reaction of acetone which includes the following steps: (a) feeding an adiabatic reactor from top to bottom, with a feed stream having a gaseous current with hydrogen and a liquid current with fresh acetone; (b) passing the feed stream of step (a) through a fixed catalytic bed, contained inside the reactor to obtain a reactor outlet effluent including liquid and gaseous phases, the catalytic bed operating in a trickle-flow regime and including at least one metallic copper based catalyst; (c) separating the effluent leaving the reactor by dividing the liquid phase from the gaseous phase; (d) recycling a part of the liquid phase from step (c), reintroducing the part, named recycling stream, into the liquid current of step (a), whereby the ratio between the mass flow rates of the recycling stream and fresh acetone, named recycling ratio, satisfies the equation.

### CROSS-REFERENCE TO RELATED APPLICATIONS

This application is a 35 U.S.C. § 371 National Stage patent application of PCT/IB2023/062188 filed 4 Dec. 2023, which claims the benefit of Italian patent application no. 102022000025110, filed on 6 Dec. 2022, the disclosures of which are incorporated herein by reference in their entirety.

### TECHNICAL FIELD

The present disclosure refers to a process for preparing isopropanol in high yields and with a high degree of purity.

### BACKGROUND

Isopropanol, commonly called isopropyl alcohol (IPA), is widely used in numerous sectors, for example as a solvent and as a reagent in laboratory reactions and on an industrial scale, as a disinfectant in hygiene products, as a preservative in cosmetic products, as an additive in paints, dyes and inks, as an antifreeze in fuels.

Said alcohol is used, in most of the uses listed above, with a high degree of purity, often higher than 99.0%.

One of the processes used industrially to produce isopropanol comprises the reaction of reduction of the acetone in the presence of hydrogen and metallic catalysts.

One of the industrial solutions used in this type of reaction consists in using fixed bed reactors, which operate in a heterogeneous phase, where the reagents, the acetone and the hydrogen, enter the reactor inside which the catalyst is immobilised. Said catalyst, generally composed of metallic particles, is dispersed on an inert or reactive support in such a way that, by expanding the area of the catalytic surface, the activity thereof and its selectivity can be increased, minimizing costs. In fact, by implementing said operational solution, the metallic active components, which are often expensive, are used in small quantities.

Since the reduction of the acetone to isopropanol is an exothermic reaction, characterized by an equilibrium enthalpy equal to −13.4 kcal/mole, if not controlled, it can lead to reaching in the reactor high temperatures, which can generate side reactions both of the reagent and of the product, resulting in the formation of undesired products. Due to its low boiling point, the acetone evaporates easily and the presence of an excess thereof in the gaseous state can lead to the formation of warmer reaction zones, even near the catalytic bed, where the process of formation of the by-products is accelerated by the presence of the catalyst. Furthermore, said by-products, by also coming into contact with the hydrogen and with the catalyst, lead to obtaining further undesired products, thereby causing a further lowering of the selectivity of the reaction. The main by-products of the hydrogenation reaction of acetone are methyl isobutyl ketone (MIBK), methyl isobutyl carbinol (MIBC), and 2-methyl-2,4-pentanediol (HEG). From the aldol condensation between two acetone molecules, diacetone alcohol (DAA) is obtained as first by-product and which near the catalytic bed can react with hydrogen to give 2-methyl-2,4-pentanediol (HEG) or, due to the reaction temperatures, generally higher than 60° C., can dehydrate to give mesityl oxide (MOX). Said oxide being reactive reacts very quickly with hydrogen to give methyl isobutyl ketone (MIBK), which is subsequently in turn reduced to methyl isobutyl carbinol (MIBC). The formed isopropanol, on the other hand, in the presence of high temperature zones, can react with itself and lead to the formation of diisopropyl ether (DIPE). In the Diagram (I) reported below, the main by-products and the secondary reactions described above that lead to their formation are illustrated.

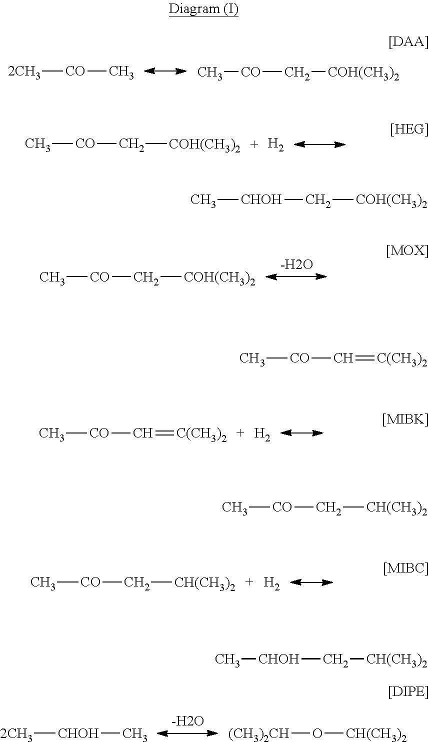

To decrease the formation of undesired products, the catalytic hydrogenation reaction can be carried out at temperature values comprised between 60-90° C., where 60° C. is the minimum trigger temperature.

By operating at these temperatures, the catalytic hydrogenation of acetone requires very long times and the use of high amounts of catalyst to achieve completion, thus compromising the economy of the process. Consequently, working at low temperatures, to obtain an isopropanol with the high degree of purity required by the various industrial applications, with low quantities of unreacted acetone and using an economically convenient process, it is necessary to implement either expensive purification processes, which lead to a decrease in the process yield and to an increase in the total production costs, or different reactor solutions, sometimes not applicable at the industrial level due to the complexity of the same.

There are patents in the literature that describe various methods for optimizing the isopropanol synthesis process starting from the catalytic hydrogenation reaction of the acetone.

In EP1070698 and EP2207762 Ineos, to decrease the by-products of the acetone reduction reaction, described a technical solution using two stages. Said solution is composed of two reactors in series operating at different temperatures and pressures: in the first one, a large part of the acetone present in the reaction mixture is reduced, while in the second one, which can be considered a finisher, the reaction is generally carried out at a lower temperature than that of operation of the first one, to increase its yield and decrease the formation of the by-products. In particular, in EP2207762 a cooling system is provided between the first reactor and the finisher, capable of bringing the temperature in the initial part of the finisher to a value comprised between 60-90° C., preferably between 60-80° C.

In EP1444184 Shell describes a multi-tubular reactor to optimize both the acetone conversion and the hydrogenation reaction yield. The multi-tubular system allows a better control over the reaction heats. The catalyst used is based on Nickel oxide or Raney Nickel.

Mitsui in EP0379323 describes the hydrogenation of the acetone to isopropanol carried out in a trickle-flow reactor, in which mainly Nickel-based catalysts are used, as described extensively in the experimental part. The trickle-flow reactors are multi-phase systems consisting of a fixed bed catalyst that is traversed by a stream of reactants present simultaneously in the liquid and gaseous state. The gaseous phase can flow upwards or downwards depending on the type of application. The liquid phase, however, primarily flows or “drips” onto the solid catalyst in a downward, top-down direction. The same word “Trickle” describes the operating characteristics in which the liquid flows intermittently on the solid catalyst in the form of films, trickles or droplets.

In EP0361755 Mitsui in the description deals generically with the hydrogenation reaction of the acetone within the phenol production process in the absence of by-products. As reported in EP0379323 in the experimental part the hydrogenation reaction carried out in a fixed bed reactor in the presence of Nickel Raney as a catalyst is described.

Finally, in EP2202214, Mitsui declares that, an acid treatment carried out on the Nickel Raney catalyst, washing it with an acidic aqueous solution before carrying out the hydrogenation reaction of acetone, can improve the conversion of the ketone and the selectivity of the alcohol obtained, as it reduces the traces of impurities linked to the catalyst system.

In the examples of the patents cited, the catalysts used to carry out this type of reaction are based on Nickel or are Raney Nickel type catalysts. The latter is usually activated in situ starting from a Nickel-based catalyst, through the addition of soda. Said activation process entails the use of washing water in the catalytic bed to make the reaction environment neutral and eliminate its alkalinity. The presence of an alkaline environment would be harmful in this type of transformations, as it would increase the formation of by-products due, for example, to the self-condensation of the acetone itself, such as the diacetone alcohol (DAA). By-products that by presenting unsaturations would in turn be reduced by the excess hydrogen present, as reported in Diagram I. The confirmation of this comes precisely from the solution provided by Mitsui, which in EP2202214 provides, in order to improve the yield and the reaction selectivity, for an acid treatment of the Raney Nickel-based catalyst before carrying out the reduction.

All these treatments, in addition to being economically expensive, introduce a certain amount of water into the reaction environment, which must then be removed from the final mixture. The removal of water in a mixture of isopropanol and possibly unreacted acetone is known to be not a simple and standardized process, as said mixture generates different azeotropes that vary with the varying concentration of the components.

In addition, the Nickel-based catalysts require special disposal procedures as they are highly polluting to the environment and toxic to humans.

The solutions cited in EP1070698 and EP2207762 by Ineos additionally provide for the use of a complex and economically disadvantageous reactor system, consisting of several reactors in series that carry out the same reaction.

It is therefore important to identify an economically advantageous process that allows, by using a simplified reactor and catalytic system, to maximize the conversion, the selectivity and the yield of the acetone reduction reaction to give isopropanol with a high degree of purity.

### SUMMARY

The present disclosure therefore provides an economically advantageous process for producing isopropanol from the catalytic hydrogenation reaction of acetone, which allows said alcohol to be obtained in high yields and with a high degree of purity, useful so as to be directly applied in the different industrial fields, without resorting to expensive purification processes or to complex reactor solutions, which are uneconomical and not applicable at an industrial level. 5 It is known, as reported by Mitsui in EP0379323, that one of the simple and economical reactor systems used in the reaction of production of isopropanol starting from acetone is characterized by a reactor having a fixed catalytic bed, operating in the trickle-flow regime with equi-current feed, from top to bottom, composed of a gaseous current containing hydrogen and a liquid current consisting of acetone, in the presence of metallic catalysts, preferably based on Nickel as described in the experimental part.

However, Mitsui exclusively defines the operating conditions that allow working in a trickle-flow regime, it does not describe the quality of the isopropanol obtained, the quantity of the by-products present in it and does not dwell on the possibility of optimizing the process by recycling the effluent leaving the reactor in a controlled manner, thus trying to maximize its productivity.

By carrying out several experimental tests, operating in a trickle-flow reactor, mainly using economical copper-based catalysts, which do not require difficult disposal procedures, recycling part of the effluent leaving the hydrogenation reactor, it could be surprisingly seen that by recycling said effluent in a certain manner, there is an operating zone in which to work that allows to simultaneously maximize the conversion, the selectivity and consequently the yield of the reaction. In this way it was possible to obtain a synthesis process of isopropanol in high yields and with a high degree of purity, by means of an economically advantageous and industrially applicable process.

### BRIEF DESCRIPTION OF THE DRAWINGS

FIG. 1: Reports the trend of the values of reaction yield (IPA yield) and of acetone conversion as a function of the recycling ratio (RR), obtained by carrying out several hydrogenation tests at values of temperature, measured at the beginning of the catalytic bed, comprised between 120-122° C., at values of mass flow rate of fresh acetone divided by the mass of the catalyst in kg (WHSV) of 1.86 h⁻¹ and at a pressure of 21 bar.

FIG. 2: Reports the trend of the values of reaction yield (IPA yield) and acetone conversion as a function of the recycling ratio (RR), obtained by carrying out several hydrogenation tests at values of temperature, measured at the beginning of the catalytic bed, comprised 83-92° C., at values of mass flow rate of fresh acetone divided by the mass of the catalyst in kg (WHSV) of 1.00 h⁻¹ and at the pressure of 21 bar.

### DETAILED DESCRIPTION OF THE DRAWINGS

All terms used in this patent application, unless otherwise indicated, are to be understood in their ordinary meaning, as known in the technical field in which they are applied.

In this patent application, the acronym “HPLC” means high performance liquid chromatography. The term “recycling effluent”, “effluent recycling”, “recycling current”, “recycling stream” means a material current that is first driven away from a main current and then is introduced back into the same current from which it had been driven away, passing through a series of chemical-physical treatments before being reintroduced.

The term “fresh acetone” means acetone introduced directly into the reactor and not coming from the recycling effluent.

The term “recycling acetone” means the acetone from the recycling effluent.

The abbreviation exp means an exponential function, which associates a value with the elevation to power having the Euler number as a base, whose first 10 digits are 2.718281828, and the number obtained from the equation in brackets as an exponent.

The term WHSV means the weight hourly space velocity, measured in h⁻¹, defined as the mass hourly flow rate of the stream of fresh acetone, i.e. without considering the recycling acetone, divided by the mass amount of the catalyst.

The temperature of the hydrogenation reaction indicated with T is defined as the temperature measured in degrees Celsius by a probe placed at the beginning of the catalytic bed present in the reactor, in the direction of the stream of the reagents, i.e. from top to bottom. This temperature coincides with the temperature of the oil passing in the jacket of the preheater (P) located before the reactor (R).

The term mass flow rate means the amount of reagent to which the flow rate measured in Kg/h is bound.

The abbreviation m_{R} means the value of the mass flow rate of the recycling stream measured in kg/h.

The abbreviation m_{A} means the value of the mass flow rate of fresh acetone measured in kg/h.

The abbreviation RR indicates the recycling ratio obtained by dividing the value of the mass flow rate of the recycling effluent m_{R} with that of the mass flow rate of fresh acetone m_{A}.

The term selectivity (S, %) means the ratio between the isopropanol moles produced and the total moles of reacted acetone multiplied by percent.

The term conversion (C, %) means the ratio between the reacted moles of acetone and the total moles of fresh acetone multiplied by percent.

The term “about” means a value within a statistical range of the order of magnitude preferably comprised between the value indicated by the measurement increased or decreased by 10%, more preferably by 5%, advantageously by 0.1%. Sometimes such a range lies in the experimental error range typical of the standard measurement methods used to measure or determine a given magnitude.

Unless otherwise reported, in the context of the present disclosure, the indication that a process comprises one or more steps means that the steps described must be absolutely part of the same, but this does not mean that other implicit steps, not directly described, can also alternatively be part of the process itself.

Unless otherwise reported, in the context of the present disclosure the indication that a current, a stream comprises one or more components means that the components described must be absolutely part of the same, but this does not mean that other implicit components, not directly described, can also alternatively be part of the same.

Unless otherwise indicated, in the context of the present disclosure a range of values indicated for a certain parameter, for example the weight or the volume of a component of a mixture, a temperature range, includes the upper and lower limits of said range and all intermediate values comprised therebetween.

The present disclosure therefore provides a process for producing isopropanol from a catalytic hydrogenation reaction of acetone comprising the following steps:
a) feeding an adiabatic reactor from top to bottom, with a feed stream consisting of a gaseous current comprising hydrogen and a liquid current comprising fresh acetone;
b) passing the feed stream of step (a) through a fixed catalytic bed, contained inside the reactor, so as to obtain a reactor outlet effluent consisting of a liquid phase and a gaseous phase, said catalytic bed operating in a trickle-flow regime and comprising at least one metallic copper based catalyst;
c) separating the effluent leaving the reactor by splitting the liquid phase from the gaseous phase;
d) recycling a part of the liquid phase obtained in step (c), reintroducing said part, named recycling stream, into the liquid current of step (a), so that the ratio between the mass flow rates of the recycling stream and fresh acetone, named recycling ratio (RR), satisfies the following equation:

$$- 1 + \frac{1}{WHSV [ {h}^{- 1} ]} ( {e}^{- \frac{4250}{T [ ° C . ] + 273.15} + 13.23} ) \leq \frac{{m}_{R}}{{m}_{A}} \leq - 1 + \frac{1}{WHSV [ {h}^{- 1} ]} ( {e}^{- \frac{4250}{T [ ° C . ] + 273.15} + 14.27} ) \qquad ( I )$$

wherein:
m_{R} is the mass flow rate of the recycling stream;
m_{A} is the mass flow rate of fresh acetone;
WHSV is the mass flow rate of fresh acetone divided by the mass of the catalyst in kg;
T is the temperature measured in Celsius degrees at the beginning of the catalytic bed present in the reactor.

The hydrogenation reaction of acetone is typically carried out in an adiabatic reactor, operating in the trickle-flow regime, equipped with an inlet and an outlet for loading and unloading reagents and products, with a tube for sampling the liquid phase and a mobile electronic probe, useful for observing the thermal profile of the reaction.

In a preferred embodiment said reactor is a tubular, steel reactor of such dimensions as to allow the loading of the catalyst in industrial size without encountering the problems related to the wall effect. Inside the reactor there is the catalytic bed, comprising the catalyst, the length of which is comprised between ⅓ and ⅔, preferably said length is about ½ of the total length of the reactor. For the entire length of said catalytic bed there is a sheath of about 2 mm thick inside which there is a mobile electronic probe, which detects the thermal profile of the bed itself. Said catalytic bed is packed between two layers of catalytically inert material. In a preferred embodiment said inert material consists of glass balls.

The reactor is fed from top to bottom, as described in step (a), by an equi-current feed stream, consisting of a gaseous current, comprising hydrogen, and a liquid current comprising fresh acetone. In a preferred embodiment, before entering the reactor, the fresh acetone is loaded into a tank and subsequently, with the use of an HPLC pump, is suitably mixed first with the recycling stream of step (d), then with the gaseous current comprising hydrogen, both dosed with appropriate thermo-mass flow rate regulators.

The gaseous current comprises hydrogen, it may also contain other inert gases, such as for example nitrogen, helium and argon. Particularly preferred is the embodiment wherein the gaseous current is composed only of hydrogen.

Being the reactor adiabatic, the feed stream of step (a) is preheated by a preheater and by an electrical belt, both located upstream of the reactor itself. The preheater is heated by the oil, present in its outer jacket, whose temperature is set to the same value as the temperature measured at the beginning of the catalytic bed. The electrical belt, placed in the tube that connects said preheater to the reactor itself, instead is preferably heated to a higher temperature by a value comprised between 3-5 C compared to the value of the temperature measured at the beginning of the catalytic bed. The temperature values of the preheater and of the thermal belt are set in such a way that at the beginning of the catalytic bed the desired temperature value is reached.

Subsequently, inside the reactor, the feed stream of step (a) comes into contact with the fixed catalytic bed, step (b), where the hydrogenation reaction takes place, said bed, operating in the trickle-flow regime and comprising at least one copper-based catalyst.

In a preferred embodiment said at least one copper-based catalyst may comprise a compound selected from copper oxide, copper chromite and mixtures thereof.

In a particularly preferred embodiment said at least one copper-based catalyst comprises copper oxide.

Other compounds or chemical elements may be present in the copper-based catalyst in minor amounts. Said compounds may be selected from alkali metal and/or alkaline earth metal oxides, transition metal oxides, aluminium trioxide, silicon dioxide and mixtures thereof.

In a preferred embodiment the catalyst comprising copper oxide may contain calcium oxide, manganese dioxide and silicon dioxide.

In another preferred embodiment the catalyst comprising copper oxide may contain zinc oxide and aluminium trioxide.

In a further embodiment the catalyst comprising copper oxide may contain aluminium trioxide.

The effluent leaving the reactor, step (c), consisting mainly of unreacted isopropanol and acetone and hydrogen, is separated by splitting the liquid phase from the gaseous phase.

In a preferred embodiment the liquid phase of the leaving effluent is separated from the gaseous phase in a phase separator, cooled to a temperature comprised between −10 and 0° C., preferably to −5° C.

Before entering the phase separator, the effluent leaving the reactor passes through a condenser placed at the same temperature as the separator.

A part of the liquid effluent leaving the separator is recycled, as reported in step (d), by reintroducing said part, named recycling stream, into the liquid current of step (a), so that the ratio between the mass flow rates of the recycling stream and fresh acetone, named recycling ratio (RR), satisfies the following equation:

$$- 1 + \frac{1}{WHSV [ {h}^{- 1} ]} ( {e}^{- \frac{4250}{T [ ° C . ] + 273.15} + 13.23} ) \leq \frac{{m}_{R}}{{m}_{A}} \leq - 1 + \frac{1}{WHSV [ {h}^{- 1} ]} ( {e}^{- \frac{4250}{T [ ° C . ] + 273.15} + 14.27} ) \qquad ( I )$$

Wherein:
m_{R} is the mass flow rate of the recycling stream;
m_{A} is the mass flow rate of fresh acetone;
WHSV is the mass flow rate of fresh acetone divided by the mass of the catalyst in kg;
T is the temperature measured in Celsius degrees at the beginning of the catalytic bed present in the reactor.

In a preferred embodiment the hydrogenation reaction is carried out at a temperature T, measured at the beginning of the catalytic bed, comprised between 7° and 140° C. Working at temperature values lower than 70° C. the reaction is not completed as we are close to the trigger limit of the same, whereas operating at temperature values higher than 140° C. greatly increases the amount of residual acetone present in the recycling stream.

In a particularly preferred embodiment, the reaction is carried out at a T comprised between 8° and 125° C.

In a preferred embodiment the values of mass flow rate of fresh acetone divided by the mass of the catalyst (WHSV) are comprised between 0.90-3.00 h⁻¹. Lower values lead to increases in point temperatures on the catalytic bed, increasing the deactivation speed of the catalyst and consequently the amount of residual acetone present in the recycling stream, while higher values do not allow to complete the reaction kinetics with, also in this case, a greater amount of residual acetone present in the effluent leaving the reactor.

In a particularly preferred embodiment, the values of mass flow rate of fresh acetone divided by the mass of the catalyst (WHSV) are comprised between 1.00-2.00 h⁻¹.

In a preferred embodiment the molar ratio between fresh acetone and hydrogen is comprised between 1.1 and 2.0. Operating with molar ratio values between fresh acetone and hydrogen lower than 1.1 results in lower conversions and increases in point temperatures on the catalytic bed that promote the catalyst deactivation process are favoured, whereas working with molar ratio values between fresh acetone and hydrogen higher than 2 increases the amount of unreacted hydrogen with consequent aggravation of the process costs.

The plant is always kept under pressure to favour the complete condensation of acetone and of the light components formed in reaction. The optimal pressure values applied in the present disclosure are determined by the excess hydrogen and are lower than 30 bar.

In a preferred embodiment the pressure of the plant is comprised between 18-25 bar, advantageously between 20-21 bar. Working at higher pressures would favour the formation of isopropanol, but would increase the process costs of a plant operating in the trickle-flow regime.

In a particularly preferred embodiment, the hydrogenation reaction is carried out at a pressure of 21 bar, varying the values of mass flow rate of the stream of fresh acetone divided by the mass of the catalyst (WHSV) in the range comprised between 0.90 and 2.00 h⁻¹, maintaining the molar ratio between fresh acetone and hydrogen equal to 1.6 and the temperature, measured at the beginning of the catalytic bed, at values comprised between 80-122° C.

The reaction effluents, after separating the excess hydrogen, are conveyed to a tank which is periodically emptied.

Sampling, useful to determine the progress of the reaction, can be carried out with a pressurised cylinder placed after the phase separator. The samples taken are subsequently analysed on the gas-chromatograph with techniques known to the person skilled in the art to determine the yield, the selectivity of the reaction, the percentage of converted acetone and the main impurities.

Surprisingly, it has been found that, by operating at different values of temperature and mass flow rate of fresh acetone divided by the mass of the catalyst being fed (WHSV), maintaining the ratios between the mass flow rates of the recycling stream and fresh acetone, i.e. the recycling ratio (RR) values, within the range defined by equation (I), an isopropanol synthesis process is obtained which simultaneously allows to obtain isopropanol with yields higher than 99.00%, selectivity higher than 99.80% and to reach acetone conversion values higher than 99.30%, minimizing the production of the reaction by-products.

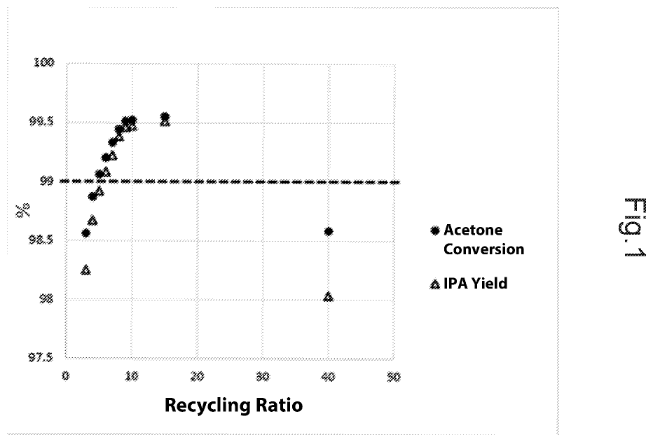

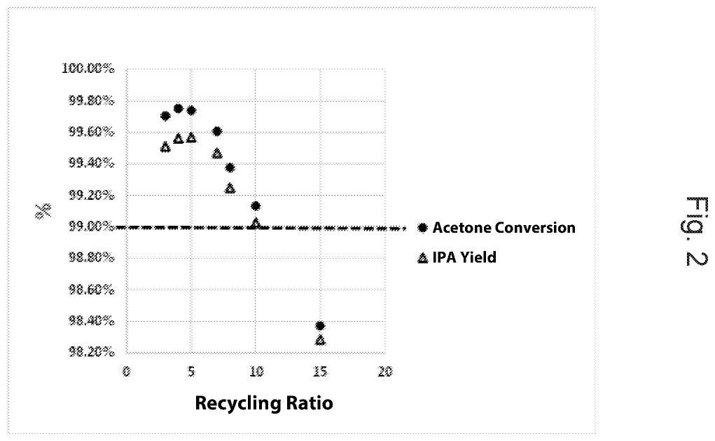

As can be noted from FIGS. 1 and 2, which describe by way of example, the trend of the acetone yield and conversion with reference to the recycling ratio (RR) in different tests carried out under different operating conditions, it is evident that these magnitudes simultaneously reach maximum values only in a certain range of recycling ratios, a range delimited by the dotted line. The values below the line are those outside the scope of the disclosure and some of these are reported in the experimental part as comparative examples. In particular, in FIG. 1, which reports the yield and the conversion of various tests carried out at values of temperature, measured at the beginning of the catalytic bed, comprised between 120-122° C., at WHSV values of 1.86 h⁻¹ and at a pressure of 21 bar, it can be noted that there are optimal values of these parameters for recycling ratios comprised between 5 and 15, in accordance with equation (I). In FIG. 2 where the reactions were carried out at values of temperature, measured at the beginning of the catalytic bed, comprised between 83-92° C., at WHSV values of 1.00 h⁻¹ and at a pressure of 21 bar, there are optimal values of these parameters for recycling ratios comprised between 3 and 10, in accordance with equation (I). For greater clarity in the graphs, the selectivity values have not been reported, which for the examples in accordance with the disclosure, reported above the dotted line, are all higher than 99.80%.

In particular, operating with recycling ratios (RR) within the range of equation (I), acetone conversion values higher than 99.30% were obtained and an isopropanol was obtained with yields higher than 99.00%, selectivity higher than 99.80%, simultaneously having a content of methyl isobutyl carbinol (MIBC) lower than 1500 ppm, of methyl isobutyl ketone (MIBK) lower than 100 ppm, of 2-methylpentane-2,4-diol (HEG) lower than 1500 ppm and a level of other impurities lower than 1000 ppm, provided that the total sum of all impurities is lower than 2500 ppm, preferably lower than 2000 ppm. Said other impurities, the total sum of which is reported respectively in Tables 1, 2, 3 and 4 of the experimental part, are preferably selected from methanol, n-propanol, methylcyclopentane, 2-methyl-1-propanol, diisopropylether (DIPE), cyclohexane, 2-methyl-1-butanol, diacetone alcohol (DAA).

On the other hand, by carrying out the reaction with ratio values between the mass flow rates of the recycling stream and fresh acetone, i.e., the recycling ratio (RR) values, outside the range defined by equation (I), as described in the comparative examples reported in the experimental section, acetone conversion values lower than 99.00%, isopropanol selectivity values lower than 99.80% and isopropanol yield values lower than 99.30% are obtained. Although in some comparative examples high selectivity values are obtained, around 99.70% for example, a decrease of about one percentage point in the respective yield and conversion values is noted. This demonstrates that working outside the range defined by equation (I) does not maximize both selectivity, conversion, and yield values. In addition, by operating under these conditions on average an isopropanol with a total impurity content greater than 2000 ppm, often higher than 2500 ppm is obtained.

In this way it is always possible to define a range of recycling ratios at different operating conditions of temperature and partial hourly speed of fresh acetone being fed (WHSV), to try to maximize the yield, the conversion, the selectivity of the reaction.

The following experimental section describes in detail the process of the disclosure, by way of non-limiting example only.

### EXPERIMENTAL SECTION

### Example 1

60 g of BASF Cu-0560 copper is loaded into a tubular steel reactor 70 cm long and 1.57 cm (¾ inch) in inner diameter to form a catalytic bed of about 35.5 cm in length.

The reactor is subsequently fed continuously from top to bottom by a stream consisting of hydrogen and acetone in such a way that the molar ratio between hydrogen and fresh acetone is equal to 1.6. The WHSV of acetone being fed is equal to 1.86 h⁻¹.

The temperature, measured at the beginning of the catalytic bed, is 89.1° C. and the operating pressure of the plant is 21 bar.

A part of the liquid effluent leaving the reactor generates the recycling stream, which is added as a feed to the starting fresh acetone. With a recycling stream calculated to work with a recycling ratio (RR) value equal to 3, which value satisfies the conditions of equation (I), isopropanol is obtained with a yield of 99.00% and a selectivity of 99.80%. Acetone conversion is around 99.30%.

### Examples 2-4

Several acetone hydrogenation tests were carried out following the procedure described in Example 1, working at values of temperature, measured at the beginning of the catalytic bed, indicated in Table 1, comprised between 100 and 101 C and maintaining the values of the molar ratio between hydrogen and fresh acetone, of WHSV and of the operating pressure of the plant unchanged.

Operating with recycling ratio (RR) values, which satisfy equation (I), comprised between 2 and 9, the reaction takes place with yields greater than 99.25% and selectivity higher than 99.80%. Acetone conversion is equal to or greater than 99.40% (Table 1).

TABLE 1

| | | | | | | | | | | | Other |
|---|---|---|---|---|---|---|---|---|---|---|---|
| | Batch | WHSV | T | | | | | MIBK | MIBC | HEG | impurities |
| Example | number | [h\hat{ }−1] | [° C.] | RR | C % | S % | Yield % | [ppm] | [ppm] | [ppm] | [ppm] |
| 2 | 20418/45 | 1.86 | 100.1 | 5 | 99.59 | 99.84 | 99.43 | 17 | 404 | 1050 | 505 |
| 3 | 20418/38 | 1.86 | 100.2 | 7 | 99.40 | 99.89 | 99.28 | 11 | 189 | 991 | 284 |
| 4 | 20418/46 | 1.86 | 100.6 | 6 | 99.57 | 99.82 | 99.40 | 19 | 367 | 1141 | 560 |

### Examples 5-9

Several acetone hydrogenation tests were carried out following the procedure described in Example 1, working at values of temperature, measured at the beginning of the catalytic bed, indicated in Table 2, comprised between 120-122 C and maintaining the values of the molar ratio between hydrogen and acetone, of WHSV and of the operating pressure of the plant unchanged.

Operating with recycling ratio (RR) values comprised between 5 and 16, which satisfy equation (I), the reaction takes place with selectivity higher than 99.85% and yields greater than 99.25%. Acetone conversion is greater than 99.35% (Table 2).

TABLE 2

| | | | | | | | | | | | Other |
|---|---|---|---|---|---|---|---|---|---|---|---|
| | Batch | WHSV | T | | | | | MIBK | MIBC | HEG | impurities |
| Example | Number | [h\hat{ }−1] | [° C.] | RR | C % | S % | Yield % | [ppm] | [ppm] | [ppm] | [ppm] |
| 5 | 20418/51 | 1.86 | 120.8 | 7 | 99.38 | 99.89 | 99.27 | 24 | 581 | 366 | 467 |
| 6 | 20418/10 | 1.86 | 121.0 | 8 | 99.44 | 99.94 | 99.38 | 8 | 375 | 301 | 291 |
| 7 | 20418/11 | 1.86 | 121.4 | 9 | 99.51 | 99.95 | 99.46 | 5 | 289 | 290 | 254 |
| 8 | 20418/12 | 1.86 | 121.4 | 10 | 99.52 | 99.95 | 99.47 | 4 | 241 | 294 | 284 |
| 9 | 20418/28 | 1.86 | 121.6 | 15 | 99.55 | 99.96 | 99.51 | 11 | 137 | 262 | 293 |

### Examples 10-17

Several acetone hydrogenation tests were carried out, following the procedure described in Example 1, with WHSV values equal to 1.00 h⁻¹ and working at values of temperature, measured at the beginning of the catalytic bed, indicated in Table 3, comprised between 83-92° C.

As can be noted from the results reported in Table 3, selectivity values higher than 99.80%, acetone conversion equal to and higher than 99.70% and yields higher than 99.50%, by working with recycling ratio (RR) values, which satisfy equation (I), comprised between 2 and 12 are obtained.

TABLE 3

| | | | | | | | | | | | Other |
|---|---|---|---|---|---|---|---|---|---|---|---|
| | Batch | WHSV | T | | | | | MIBK | MIBC | HEG | impurities |
| Example | Number | [h\hat{ }−1] | [° C.] | RR | C % | S % | Yield % | [ppm] | [ppm] | [ppm] | [ppm] |
| 10 | 20428/81 | 1.00 | 83.7 | 3 | 99.70 | 99.81 | 99.51 | 1 | 575 | 1358 | 332 |
| 11 | 20428/80 | 1.00 | 84.6 | 4 | 99.75 | 99.81 | 99.56 | <I. | 295 | 1634 | 305 |
| 12 | 20428/79 | 1.00 | 86.0 | 5 | 99.74 | 99.83 | 99.57 | 2 | 152 | 1588 | 299 |
| 13 | 20428/78 | 1.00 | 87.5 | 7 | 99.61 | 99.86 | 99.47 | 1 | 70 | 1330 | 308 |
| 14 | 20445/52 | 1.00 | 89.3 | 7 | 99.84 | 99.87 | 99.71 | <I. | 131 | 784 | 675 |
| 15 | 20437/41 | 1.00 | 90.6 | 7 | 99.90 | 99.92 | 99.82 | <I. | 141 | 693 | 268 |
| 16 | 20445/50-1 | 1.00 | 90.8 | 7 | 99.87 | 99.82 | 99.68 | <I. | 344 | 894 | 938 |
| 17 | 20445/50 | 1.00 | 91.8 | 7 | 99.88 | 99.86 | 99.73 | 3 | 475 | 929 | 365 |

### Comparative Examples 18-36

Several acetone hydrogenation tests were carried out following the procedure described in Example 1, with WHSV values comprised between 1.00 and 3.72 h⁻¹, bringing the temperature, measured at the beginning of the catalytic bed, to the values indicated in Table 4, which are comprised between 8° and 121° C. and by maintaining the recycling ratio values outside the upper and lower limits of the range obtained by equation (I). It has been seen that operating with recycling ratios that do not satisfy equation (I), acetone conversion values lower than 99.00% are obtained. The selectivity of the reaction is equal to or lower than 99.80% and the yield is lower than 99.00%. In particular, it has been seen that these values either decrease all at the same time or if only the selectivity remains acceptable and around 99.70%, the conversion and yield values are below 98.00%. Table 4 reports the yields, the conversions and the selectivities of the tests carried out.

TABLE 4

| | | | | | | | | | | | Other |
|---|---|---|---|---|---|---|---|---|---|---|---|
| | Batch | WHSV | T | | | | | MIBK | MIBC | HEG | impurities |
| Example | number | [h\hat{ }−1] | [° C.] | RR | C % | S % | Yield % | [ppm] | [ppm] | [ppm] | [ppm] |
| 18 | 20418/33 | 1.86 | 80.6 | 7 | 96.57 | 99.59 | 96.17 | 128 | 524 | 3339 | 287 |
| 19 | 20418/37 | 1.86 | 90.1 | 7 | 97.84 | 99.77 | 97.62 | 44 | 299 | 1926 | 297 |
| 20 | 20418/35 | 1.86 | 90.4 | 7 | 97.97 | 99.78 | 97.75 | 34 | 271 | 1829 | 320 |
| 21 | 20418/74 | 3.72 | 89.9 | 7 | 29.83 | 91.42 | 27.27 | 5 | 314 | 17132 | 8473 |
| 22 | 20418/9 | 1.86 | 119.8 | 3 | 98.56 | 99.69 | 98.25 | 134 | 2693 | 145 | 417 |
| 23 | 20418/8 | 1.86 | 120.1 | 4 | 98.87 | 99.80 | 98.68 | 47 | 1660 | 204 | 370 |
| 24 | 20428/89 | 1.86 | 121.1 | 40 | 98.58 | 99.44 | 98.03 | 15 | 50 | 223 | 5577 |
| 25 | 20428/88 | 1.86 | 101.3 | 40 | 93.71 | 99.76 | 93.49 | 1 | 178 | 1972 | 392 |
| 26 | 20418/75 | 3.72 | 120.4 | 7 | 91.99 | 99.62 | 91.64 | 2 | 1305 | 1539 | 947 |

1) A process for producing isopropanol through a catalytic hydrogenation reaction of acetone, the process including the following steps:
a) feeding an adiabatic reactor from top to bottom, with a feed stream including a gaseous current comprising hydrogen and a liquid current comprising fresh acetone;
b) passing the feed stream of step (a) through a fixed catalytic bed, contained inside the reactor, so as to obtain a reactor outlet effluent including a liquid phase and a gaseous phase, said catalytic bed operating in a trickle-flow regime and comprising at least one metallic copper based catalyst;
c) separating the effluent leaving the reactor by splitting the liquid phase from the gaseous phase; and
d) recycling a part of the liquid phase obtained in step (c), reintroducing said part, named recycling stream, into the liquid current of step (a), so that the ratio between the mass flow rates of the recycling stream and fresh acetone, named recycling ratio, satisfies the following equation:
$$- 1 + \frac{1}{WHSV [ {h}^{- 1} ]} ( {e}^{- \frac{\text{?}}{\text{?}} + 13.23} ) \leq \frac{{m}_{R}}{{m}_{\text{?}}} \leq - 1 + \frac{1}{WHSV [ {h}^{- 1} ]} ( {e}^{- \frac{\text{?}}{\text{?}} + 14.27} ) \qquad ( I )$$
$$\text{?} \text{indicates text missing or illegible when filed}$$
wherein:
m_{R} is the mass flow rate of the recycling stream;
m_{A} is the mass flow rate of fresh acetone;
WHSV is the hourly mass flow rate of the fresh acetone stream divided by the mass of the catalyst in kg;
T is the temperature measured in Celsius degrees at the beginning of the catalytic bed present in the reactor.

2) The process according to claim 1, wherein the temperature measured at the beginning of the catalytic bed varies in a range of values comprised between 70° C. and 140° C.

3) The process according to claim 2, wherein said temperature varies in a range of values comprised between 80° C. and 125° C.

4) The process according to claim 1, wherein the hourly mass flow rate of the fresh acetone stream divided by the mass of the catalyst (WHSV) varies in a range of values comprised between 0.9 h⁻¹ and 3.0 h⁻¹.

5) The process according to claim 4, wherein said hourly mass flow rate divided by the mass of the catalyst (WHSV) varies in a range of values comprised between 1.0 h⁻¹ and 2.0 h⁻¹.

6) The process according to claim 1, wherein in step (a) the molar ratio between fresh acetone and hydrogen is comprised between 1.1 and 2.0.

7) The process according to claim 1, wherein the feed stream of step (a) is obtained by mixing the gaseous current with the liquid current including fresh acetone and the recycling stream of step (d).

8) The process according to claim 7, wherein the feed stream of step (a) is preheated before entering the reactor to a temperature value 3-5° C. higher than the temperature T measured at the beginning of the catalytic bed.

9) The process according to claim 1, wherein the gaseous current of step (a) including hydrogen.

10) The process according to claim 1, wherein in step (b) said at least one metallic copper based catalyst comprises a compound selected from copper oxide, copper chromite and mixtures thereof.

11) The process according to claim 10, wherein said at least one metallic copper based catalyst comprises copper oxide.

12) The process according to claim 1, wherein step (c) is carried out at a temperature comprised between −10° C. to 0° C.

13) The process according to claim 1, wherein the hydrogenation reaction is carried out at a pressure comprised between 15-25 bars.

14) The process according to claim 1, wherein the hydrogenation reaction is carried out at a pressure of 21 bar, varying the hourly values of mass flow rate of the fresh acetone stream divided by the mass of the catalyst (WHSV) in the range comprised between 0.90 h⁻¹ and 2.00 h⁻¹, maintaining the molar ratio between fresh acetone and hydrogen equal to 1.6 and the temperature measured at the beginning of the catalytic bed at values comprised between 80° C.-122° C.
